(19) United States
(12) Patent Application Publication
(10) Pub. No.: US 2008/0034446 A1
(43) Pub. Date: Feb. 7, 2008

(54) METHOD AND SYSTEM FOR PRODUCING BEEF CONSISTENTLY HAVING AN ELEVATED INTRAMUSCULAR FAT CONTENT

(75) Inventor: Robert Rebholtz,, Boise, ID (US)

Correspondence Address: BLACK LOWE & GRAHAM, PLLC, 701 FIFTH AVENUE, SUITE 4800, SEATTLE, WA, 98104, US

(73) Assignee: Agri Beef Co., Boise, ID (US)

(21) Appl. No.: 11/461,936

(22) Filed: Aug. 2, 2006

(51) Int. Cl. A01K 67/027

(52) U.S. Cl. 800/15

(57) ABSTRACT

The present invention comprises a system and methods for generating beef. In one embodiment, a method of breeding cattle includes selecting a male bovine having a Wagyu composition, selecting a female bovine having a composition derived from another breed and producing an offspring. In another embodiment, a data system includes a remote station positioned proximate to a cattle area to identify a selected bovine and to receive information, and a central station to exchange information with the remote station. In another embodiment, a method of generating cattle includes identifying a male bovine having a heritable genetic trait derived from the Wagyu breed, identifying a suitable female bovine, and introducing a reproductive material derived from the male bovine into the female bovine. In some embodiments, an omega-3 enriched ration is fed to the cattle during a period prior to slaughter corresponding to the weight of the cattle.

### FIELD OF THE INVENTION

This invention relates generally to animal genetics and husbandry, and in particular, to the production of beef of enhanced quality.

### BACKGROUND OF THE INVENTION

In recent years, the U.S. beef industry has steadily moved towards the production of leaner grades of beef, due at least in part to public health concerns related to foods having a high saturated fat content. As a consequence, beef consumers typically encounter a beef product that is less desirable with respect to texture, tenderness and flavor than was generally available some years ago. More recent research has indicated that the consumption of beef products having a somewhat higher saturated fat content may not be as deleterious to cardiac health as previously believed, though saturated fat is generally less healthy than other fats such as omega-3 fatty acids. Consumer preferences have begun to favor beef products that are more palatable in terms of texture, tenderness and flavor. Factors that correlate with consumer satisfaction are the fat content and the fat distribution present in the beef product. The fat distribution is generally referred to as “marbling”, and relates to the relative amount of intramuscular fat in the beef product.

Beef products are classified according to standards developed by the U.S. Department of Agriculture (USDA), and may be segregated into one of eight different quality grades. Beginning with the highest and continuing to the lowest, the eight quality grades are Prime, Choice, Select, Standard, Commercial, Utility, Cutter and Canner. The characteristics that are used to classify beef include color, texture, firmness and also the degree of marbling in the beef product, which is determined by a visual inspection of the carcass by a certified USDA inspector. Well-marbled beef generally contains substantial amounts of intramuscular fat relative to muscle, and is classified as Prime if the intramuscular fat content is about eight percent by volume, or greater. Beef having somewhat less marbling is usually classified as Choice if it contains between approximately four and eight percent intramuscular fat content by volume. Still lower amounts of marbling may result in a classification of Select, or even lower grades.

Although consumer preferences now favor the production of beef products that qualify for classification as Prime, or even the elevated classification of “Prime-plus”, the percentage of beef produced by conventional livestock herds that qualifies as Prime or Prime-plus is relatively low. One reason is that breeding practices have for some time favored the production of beef having a lower overall fat content. Consequently, conventional livestock herds tend to lack a strong genetic predisposition to produce beef at enhanced levels that may be classified as Prime or Prime-plus. In addition, conventional husbandry practices are generally not tailored and/or sufficiently controlled to promote the production of livestock that yields a high percentage of beef product that qualifies as Prime or higher. For example, the reproductive activity of a livestock herd may be inadequately managed so that the size of the herd fails to increase at an optimum rate. Moreover, elevated numbers of offspring having undesired traits may be obtained when the herd is inadequately managed. Health protocols established for the livestock herd may not necessarily favor the production of a beef product that qualifies as Prime or higher, or they may be poorly controlled.

Still other issues related to husbandry practices may also inhibit the production of Prime or Prime-plus beef. For example, the livestock herd may be fed materials that are not conducive to the generation of a Prime or higher beef product, and/or the herd may be nourished according to a schedule that inhibits the generation of a beef product having the desired classification. For example, if the herd is nourished on rangeland, the diet may be varied and include various grasses and brush that may be insufficient in nutritional content, or variable in quality. Even in cases where the livestock herd is maintained in a managed confinement facility (e.g., a feedlot), the rations may still not constitute feed of adequate quality or consistency.

Accordingly, what is needed is a system and method for consistently generating a beef product having a generally higher intramuscular fat content. It would be a further advancement in the art to generate a beef product high in healthier fats such as omega-3 fatty acids.

### SUMMARY OF THE INVENTION

The present invention comprises a system and methods for generating beef of enhanced quality. In one preferred implementation, a method of breeding beef cattle having an elevated intramuscular fat content includes selecting a male bovine having a substantially Wagyu genetic composition and further selecting a female bovine having a genetic composition derived from selected other breeds. More preferably, the cattle are selected in order to produce offspring of about 51% or greater Wagyu lineage. The method further includes producing an offspring having the elevated intramuscular fat content by combining the genetic composition of the male bovine with the genetic composition of the female bovine.

In another preferred aspect, a method of breeding beef cattle includes identifying a male bovine having a heritable genetic trait derived from the Wagyu breed, and identifying a female bovine having a heritable genetic trait derived from selected other breeds. The method further includes producing an offspring by combining a genetic material of the male bovine with a genetic material of the female bovine.

In still another preferred aspect, a data system for producing cattle having an elevated intramuscular fat content includes a remote station positioned proximate to a cattle management area that is operable to identify a selected bovine and to receive information obtained from the selected bovine, and a central station operable to exchange information with the remote station.

In still yet another preferred aspect, a method of generating beef cattle includes identifying a suitable male bovine having a heritable genetic trait derived from the Wagyu breed, and identifying a suitable female bovine. The method further includes introducing a reproductive material derived from the male bovine into the female bovine to produce an offspring having the desired intramuscular fat content.

In another preferred aspect, a method of nourishing cattle includes feeding a daily ration of omega-3 enriched feed to the cattle during a finishing period having a duration corresponding to the weight of the cattle. Most preferably, this feeding regimen is applied to cattle offspring of the type described above.

### BRIEF DESCRIPTION OF THE DRAWINGS

The preferred and alternative embodiments of the present invention are described in detail below with reference to the following drawings.

FIG. 1 is a partial diagrammatic view of a method of breeding beef cattle that produce elevated levels of intramuscular fat in a beef product, according to an embodiment of the invention;

FIG. 2 is a schematic view of a data management system for compiling and accessing data for the breeding method of FIG. 1, according to another embodiment of the invention;

FIG. 3 is a flowchart that depicts a method for generating the offspring of FIG. 1 according to still another embodiment of the invention;

FIG. 4 is a flowchart of a preferred feeding program; and

FIG. 5 is a process flow diagram of a method for generating beef having high omega-3 fatty acid content.

### DETAILED DESCRIPTION OF THE INVENTION

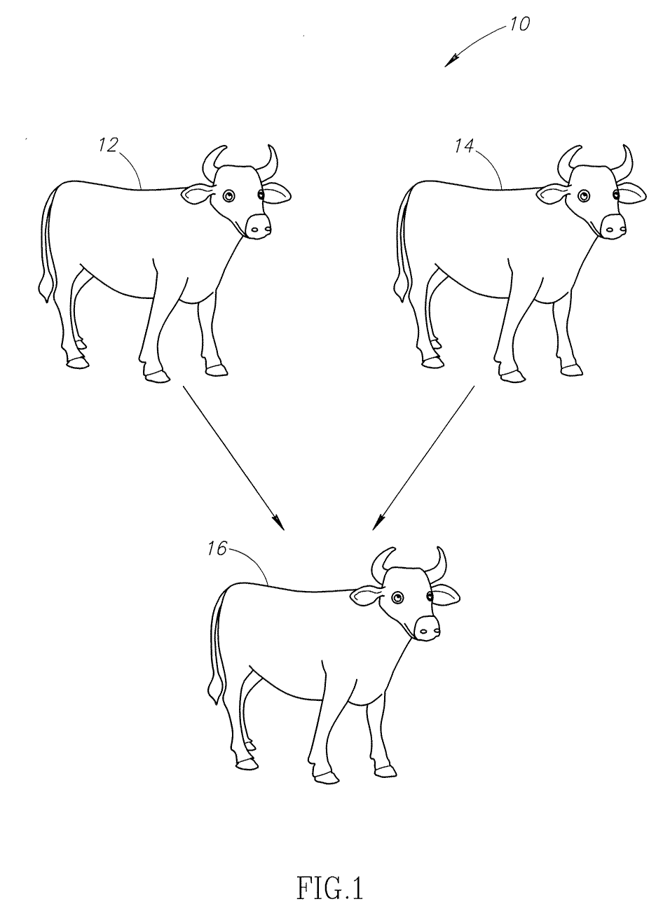

The present invention relates to production systems and methods for generating beef of enhanced quality. Many specific details of certain embodiments of the invention are set forth in the following description and in FIGS. 1 through 5 to provide a thorough understanding of such embodiments. The present invention may have additional embodiments and the present invention may be practiced with various combinations of the features described in the following description.

FIG. 1 is a partial diagrammatic view of a method 10 of breeding beef cattle that produce elevated levels of intramuscular fat in a beef product, according to an embodiment of the invention. The method 10 includes a male bovine 12 and a female bovine 14 that cooperatively produce at least one offspring 16 through sexual reproduction. The male bovine 12 includes an animal having a high genetic potential to produce a highly marbled beef product, such as a male bovine derived substantially from the Wagyu breed. The male bovine 12 may therefore include selected members generally associated with the “black” Wagyu strains, including Tottori, Tajima, Shimane, Okayama and others, as well as selected members of the “red” Wagyu strain, such as the Kochi and Kumamoto strains. In a particular embodiment, the male bovine 12 is a Wagyu sire having a genetic composition that includes, at least in part, a Tajima Wagyu influence. In another particular embodiment, the male bovine 12 may include sires of proven reproductive capability that are members of the original importation of Wagyu cattle into the U.S. in 1993-1995. Accordingly, the male bovine 12 may include Wagyu genetic strains such as the Fukutsuru, Michifuku, Takazakura and Yasufuku strains.

The male bovine 12 may also include male offspring that result from the combination of a sire of the Fukutsuru, Michifuku, Takazakura and Yasufuku strains with a dam having the Suzutani and Okutani strains. For example, sires obtained from the Sanjirou 3 and Yudai strains may serve as the male bovine 12.

The female bovine 14 may include various well-known English varieties, such as Angus, Hereford, Short Horn, Devon, Ayrshire and others. In some embodiments, the female bovine 14 may comprise an English variety having a Wagyu influence, though this is by no means required. Alternately, a selected English breed that produces milk exhibiting an elevated butterfat content may also be used. In contrast, various breeds exhibiting a Continental genetic influence, such as Limosin, Simmental, and others are generally less preferred when selecting the female bovine 14. Similarly, various “exotic” breeds, such as Maine-Anjou and others, are also less preferred. A female bovine 14 having a bos indicus influence, such as Brahman, bos taurus, and others are similarly less preferred in the method 10.

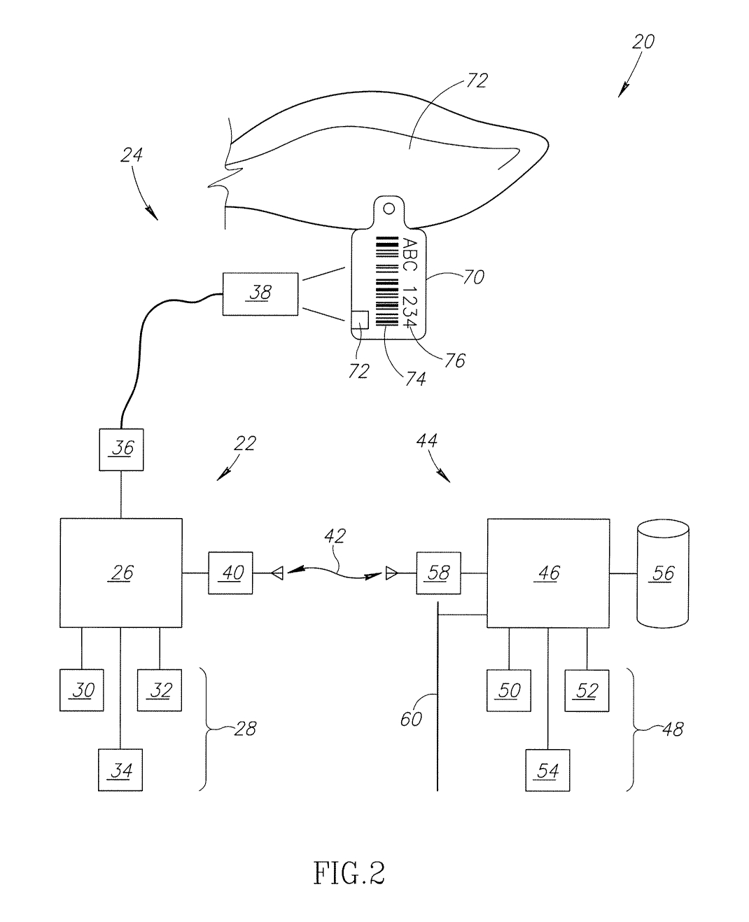

FIG. 2 is a schematic view of a data management system 20 for compiling and accessing data for the breeding method 10 of FIG. 1, according to another embodiment of the invention. The system 20 includes a remote station 22 that may be positioned proximate to an area where one or more bovine 24 are maintained. Accordingly, the remote station 22 may be located near a feedlot, a veterinary area, or other generally confined areas. The remote station 22 further includes a processing unit 26 that generally includes any programmable electronic device configured to receive programming instructions and input data, and to process the data according to the programming instructions. In one particular embodiment, the processing unit 26 is a laptop computing device, such as the Dell Inspiron 9100 Notebook, available from Dell, Inc. of Austin Tex., although other suitable alternatives exist. The processing unit 26 is coupled to a plurality of input and output devices 28, which may include a pointing device 30 operable to provide input commands to the processing unit 26, a keyboard 32 for the entry of text information and commands to the processing unit 26, and a visual display device 34 for viewing information generated by the processing unit 26. Other input and output devices 28 may include a printer (not shown) operable to generate a printed copy of information generated by the processing unit 26. The processing unit 26 further includes at least one communications port 36, such as a universal serial port (USB), FIREWIRE, or other similar communications ports. The communications port 36 is configured to receive a scanning device 38 to collect data from the bovine 24, as will be discussed in greater detail below. The processing unit 26 is also coupled to a wireless transceiver 40 that is operable to exchange wireless signals 42 with other external devices.

The system 20 also includes a central station 44 that may be positioned at a distance relative to the remote station 22. For example, the central station 44 may be positioned in an office environment. The central station 44 also includes a processing unit 46 that generally comprises a programmable electronic device configured to receive programming instructions and input data, and to process the data according to the programming instructions. The processing unit 46 is coupled to a plurality of input and output devices 48, which may include a pointing device 50 operable to provide input commands to the processing unit 46, a keyboard 52, and a visual display device 54 for viewing information generated by the processing unit 46. The processing unit 46 may also be coupled to a network 60 operable to exchange information with other processing units coupled to the network 60. The processing unit 46 is further coupled to at least one mass storage device 56 that is configured to store data acquired by the remote station 22. The mass storage device 56 may comprise a magnetic disk drive or an optical disk drive device, although other mass storage devices are well known in the art. A wireless transceiver 58 is also coupled to the processor 46 that is operable to exchange the signals 42 with the remote station 22.

With continued reference to FIG. 2, identifying a bovine 24 using the system 20 will now be described. An identification tag 70 is fixedly attached to an external portion of the bovine 24. For example, the identification tag 70 may be an ear tag that is coupled to an ear 72 of the bovine 24, as shown in FIG. 2. Alternately, the tag 70 may be a collar device configured to be positioned externally around a neck portion or a leg portion of the bovine 24. Alternately, the identification tag 70 may comprise a device configured to be positioned within a body of the bovine 24, such as a microchip device suitable for subcutaneous placement in the bovine 24. In either case, the tag 70 may include a radio frequency identification device (RFID) 72 operable to identify the bovine 24 through the stimulated emission of radio frequency energy. Accordingly, in this instance, the scanning device 38 is operable to emit radio frequency energy to stimulate the RFID 72, and to detect the corresponding and identifying emission from the RFID 72, which is, in turn, communicated to the processing unit 26. If the identification tag 70 is externally located on the bovine 24, the tag 70 may be comprised of a bar code 74 configured to be optically scanned by the scanning device 38. The identifying information present in the bar code 74 may then be transmitted to the processing unit 26. Alternately (or in addition), the tag 70 may contain an alphanumeric code 76, and the scanning device 38 suitably configured to optically scan the code 76 by means of optical character recognition (OCR).

The operation of the system 20 will now be described in detail. The system 20 may be used to compile and store information of various types relating to the bovine 24 for later retrieval. When a bovine 24 is first introduced into a population, a data file is established for the bovine 24 to document the date of birth of the bovine 24, the genetic lineage of the bovine 24, and any other relevant information. The tag 70 is also scanned to assign an identity to the data file. The data file may then be transferred to the central station 44 by means of the wireless signals 42, and stored in the database 56. As the bovine 24 develops, other information may be added to the data file in the database 56. For example, a vaccination record that documents the administration of vaccines during the lifetime of the bovine 24 may be progressively created in the system 20 by scanning the tag 70, and entering the data regarding the type of vaccine, the dosage amount, the date of vaccination and other pertinent data through the input and output devices 28. A health history may also be created by entering a description of a veterinary condition and/or treatment into the system 20 in the foregoing manner. Data files that document the reproductive history of the bovine 24 as well as feeding regimens may also be stored in the system 20.

Information stored in the database 56 of the system 20 may be communicated to similarly configured systems, or to suitably configured computational devices that are remotely located relative to the system 20 by means of the network 60. Accordingly, information may be transferred to various locations for further processing and analysis. For example, remotely located veterinary personnel may advantageously access the data stored in the system 20 in order to diagnose a veterinary condition or to develop a preventative veterinary treatment program. Other remotely located personnel may desire access to the system 20 in order to monitor a feeding program. Still other remotely located personnel may require access to information stored in the system 20 in order to monitor and/or plan reproductive activity within the bovine population.

Although FIG. 2 shows a single remote station 22 in communication with the central station 44, more than one remote station 22 may be present so that information from more than a single physical location may be documented and simultaneously transmitted to the central station 44. Moreover, although FIG. 2 depicts wireless communication between the remote station 22 and the central station 44, it is understood that various other communication links may be used. For example, the remote station 22 and the central station 44 may be coupled by a metallic conductor, a modem system, or by other similar means.

In an effort to produce beef of desirable omega-3 fat content, the introduction of certain dietary content was tested. Although omega-3 levels were somewhat influenced by dietary adjustment in a variety of cattle, surprisingly good results were obtained when applied to the Wagyu bred offspring as discussed above. Moreover, specific feeding volumes and times were found to produce ideal results while maintaining dietary efficiency.

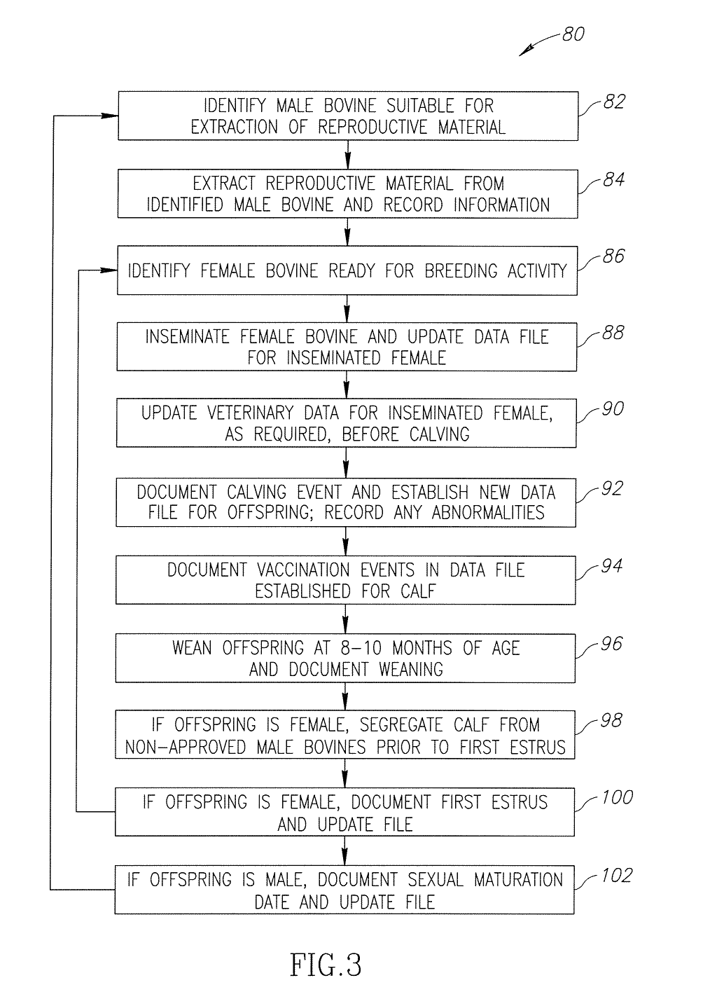

FIG. 3 is a flowchart that depicts a method 80 for generating and raising the offspring 16 of FIG. 1 according to still another embodiment of the invention. A population of beef cattle is carefully documented and managed to achieve a desirable beef product and to successfully continue the livestock production cycle. Accordingly, the method 80 preferably incorporates a suitable data management system, such as the system 20 of FIG. 3 during the implementation of the method 80. Moreover, since beef cattle are not seasonal breeders as are other animals of commercial value, such as horses, sheep and goats, for example, the method 80 may be implemented on a continuous basis through out the year.

With reference also now to FIG. 1, a male bovine 12 that is suitable for breeding is identified at block 82. The identification may be made by recourse to data files for selected individuals that are stored in the system 20 of FIG. 2. Pertinent information may include the age and genetic background of the male bovine 12, as well as any other relevant information relating to the reproductive history of the male bovine 12. At block 84, the reproductive material (semen) is removed from the selected male bovine 12 by palpation and preserved cryogenically, if desired. If preserved, the palpated semen is identified with a unique identifier prior to preservation and the data file for the selected male bovine 12 is then updated to record the unique identifier. At block 86, a female bovine 14 is identified by accessing data files established for the females within the livestock population. Relevant data in this regard may include an examination of the present reproductive status of each individual female bovine 14 in the population, the reproductive history of each female bovine 14, the preceding calving date (if any) for each bovine 14, the date of first postpartum estrus, and the actual length of the estrus cycle for each individual female bovine 14 in the population, the consistency of estrus cycle length, and the number of inseminations required to achieve pregnancy in each bovine 14. Additional information may include data concerning the incidence and severity of dystocia (if any) for each female bovine 14.

At block 88, the female bovine 14 is inseminated, using either an artificial insemination or natural method, wherein the female bovine 14 is palpated using an insemination syringe. The data file for the female bovine 14 is then updated to reflect the insemination procedure. At block 90, the female bovine 14 is subjected to periodic veterinary examinations to determine the effectiveness of the insemination procedure. If it is determined that the insemination procedure was ineffective, the data file for the female bovine 14 is updated to reflect the failed procedure, and the female bovine 14 may be subjected to the procedure at block 88 again. Alternately, if the insemination procedure is successful, the data file is updated to reflect this. If the procedure is successful, but the female bovine 14 miscarries, the data file is also updated to document this.

If the female bovine 14 produces an offspring 16, the data file for the female bovine 14 is updated to include either the actual calving date (preferred) or estimated calving date period, as well as any abnormalities associated with the calving, as shown at block 92. At this time, a data file may be established for the offspring 16. The vaccinations administered to the offspring 16, as well as other significant health maintenance milestones during the maturation of the offspring 16 may then be documented in the data file of the offspring 16, as shown at block 94.

At block 96, the offspring 16 is weaned at approximately eight to ten months of age. The weaning date, and any other information relevant to the weaning, such as the behavior of the offspring 16 following the weaning process, should be documented in the data file of the offspring 16. After weaning, the offspring 16 should be segregated according to age and gender. Female offspring 16 should be separated from all male bovine 12 not having the desired genetic background at all times following the first instance of estrus in order to avoid diluting the desired genetic traits, as shown in block 98. At block 100, if the offspring 16 is a female, the first occurrence of estrus is noted through observation of the offspring 14 and the data file for the offspring 16 is updated. After the first occurrence of estrus, the offspring 16 may be identified as eligible for breeding at block 86.

At block 102, if the offspring 16 is male, the sexual maturation of the offspring 16 is noted through observation (at approximately about 12 to 16 months) and the data file for the offspring 16 is updated. The offspring may then be identified as eligible for breeding at block 82.

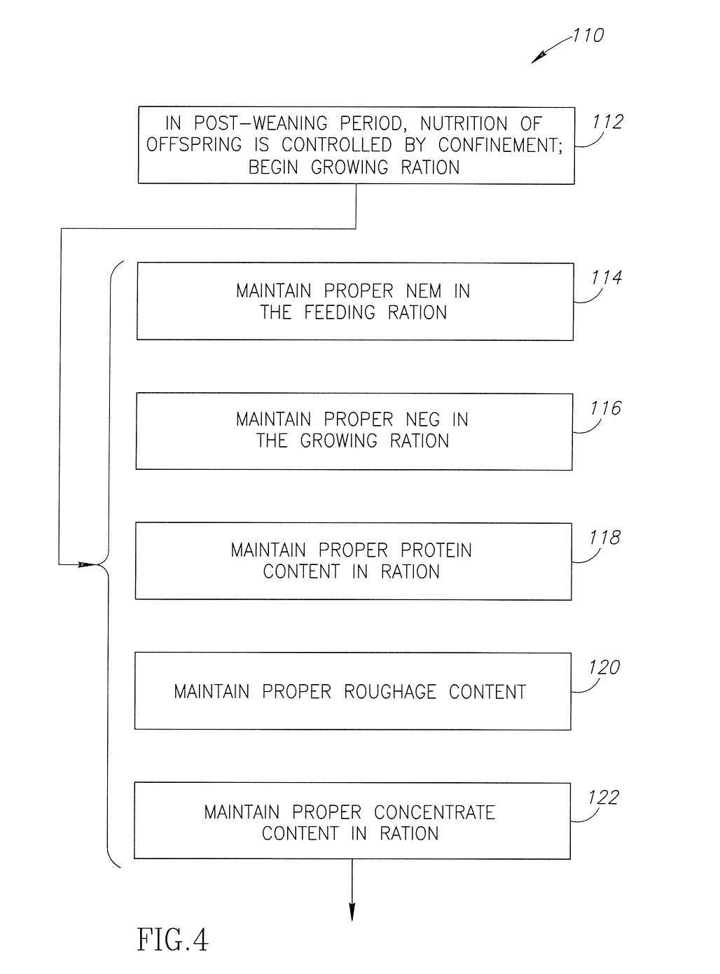

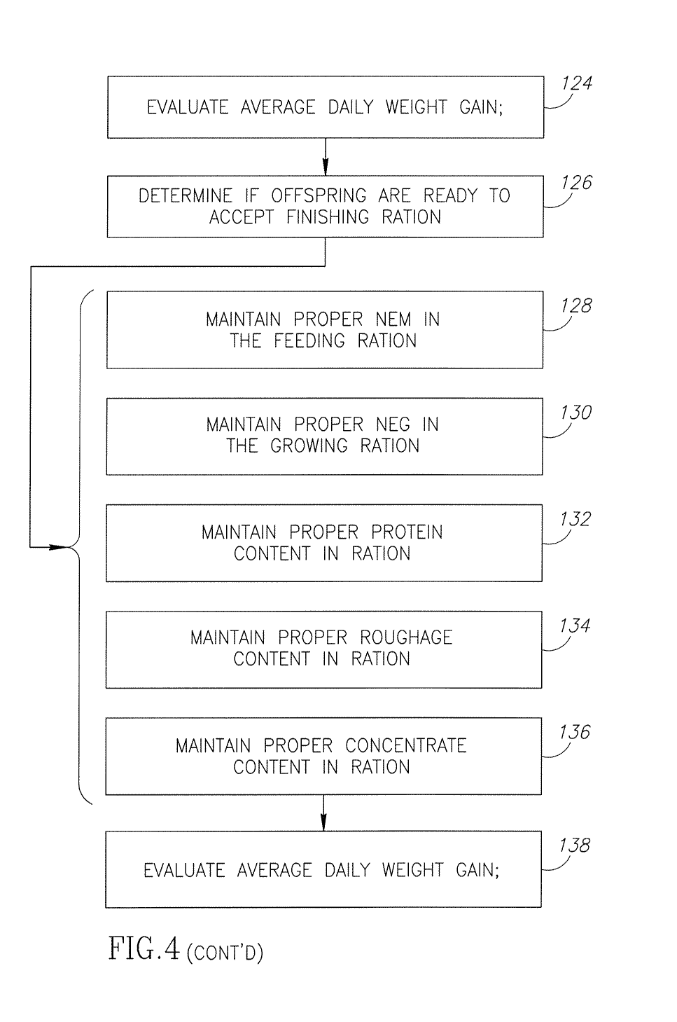

FIG. 4 is a flowchart that describes a method 110 for nourishing the offspring 16 of FIG. 1 according to yet another embodiment of the invention. In general, the quantity and quality of feeding materials may be carefully controlled in order to produce livestock having the desired characteristics in the beef product. If the nutrition is periodically inadequate, or even irregular, various bodily functions may be adversely affected. For example, the growth of offspring may be slowed, or in severe cases, completely arrested. Further, the reproductive period of female may be interrupted, thus adversely affecting the propagation of the livestock population. The method 110 is divided into two main phases. The first phase is a growing phase that follows weaning, and extends from approximately about nine months of age to approximately about thirteen months of age. The second phase is a finishing phase, and extends from about thirteen months of age to approximately fifteen months of age. During each phase, the offspring receives a total ration that may be divided into a growing ration and a feeding ration. The metabolic content in each ration may be further described as a net energy amount that contributes to the maintenance of the offspring 16 (“net energy for maintenance”, or NEM), and a net energy amount that contributes to the growth of the offspring 16 (“net energy for growth”, or NEG).

At block 112 of the method 110, the offspring 16 are confined to an enclosed area to receive nourishment, such as a feedlot or a growing lot, in order to carefully control the feed material supplied to the offspring 16. As an alternative, an enclosed pasture may be used provided that the feed material is acceptable and uniform, and is present in sufficient quantities to fully nourish the offspring 16. Starting at block 114, the nutritional components in the growing phase feeding material are described. During the growth phase, the amount of NEM in the feeding ration is between approximately about 75 and approximately about 90 MCal. In a preferred embodiment, the NEM of the feeding ration is between 80 and 85 MCal. The amount of NEG in the growing ration is between approximately about 49 and approximately about 58 MCal of the total daily ration, as shown in block 116. In a preferred embodiment, the NEG in the growing ration is between approximately about 53 and approximately about 56 MCal of the total daily ration. A protein content in the total ration ranges between approximately about 12 and approximately about 18% of the total daily ration, as shown in block 118. In a preferred embodiment, the proposition of protein in the total ration ranges between approximately about 13 and approximately about 14% of the total daily ration. A roughage component in the daily ration comprises between approximately about 15 and approximately about 27% of the total daily ration, as shown in block 120. In a preferred embodiment, the roughage component in the daily ration comprises between approximately about 18 and approximately about 23% of the total daily ration. Finally, a concentrate content in the daily ration is shown in block 122, and is between approximately about 75% and approximately about 85% of the total daily ration. In a preferred embodiment, the concentrate content is between approximately about 77% and approximately about 84% of the total daily ration. The concentrate generally comprises concentrated protein, energy and/or mineral supplements.

At block 124, the daily average weight gain for the offspring 16 is evaluated at periodic intervals, and conformity with a target of between approximately about 1.8 and approximately about 2.2 pounds per day is measured. In a preferred embodiment, the target value is approximately about 2.0 pounds per day. Based upon the accumulated weight statistics compiled at 124, a determination is made regarding the suitability of moving the offspring 16 into the finishing phase, as shown at block 126. If the offspring 16 is moved to the finishing phase, the NEM in the feeding ration ranges between approximately about 75 and approximately about 90 MCal, as shown in block 128. In a preferred embodiment, the NEM of the feeding ration is between approximately about 80 and approximately about 85 MCal. The NEG is maintained between approximately about 49 and approximately about 58 MCal of the total daily ration, as shown in block 130. In a preferred embodiment, the NEG in the growing ration is approximately about 53 and approximately about 56 MCal of the total daily ration. The protein content in the ration ranges between approximately about 12 and approximately about 18% of the total ration at block 132. In a preferred embodiment, the protein content is between approximately about 3 and approximately about 14% of the total daily ration. The roughage content in the ration is at between approximately about 15 and approximately about 27% of the total daily ration, as shown at block 134. In a preferred embodiment, the roughage component in the daily ration comprises between approximately about 18 and approximately about 23% of the total daily ration. A concentrate content in the daily ration is at between approximately about 75 and approximately about 85% of the total daily ration at block 136. In a preferred embodiment, the concentrate content of the is between approximately about 77 and approximately about 84% of the total daily ration.

The average daily weight gain for the offspring 16 is evaluated at periodic intervals during the finishing phase, and conformity with a target value of between approximately about 1.8 and approximately about 2.2 pounds per day is measured. In a preferred embodiment, the target value is approximately about 2.0 pounds per day.

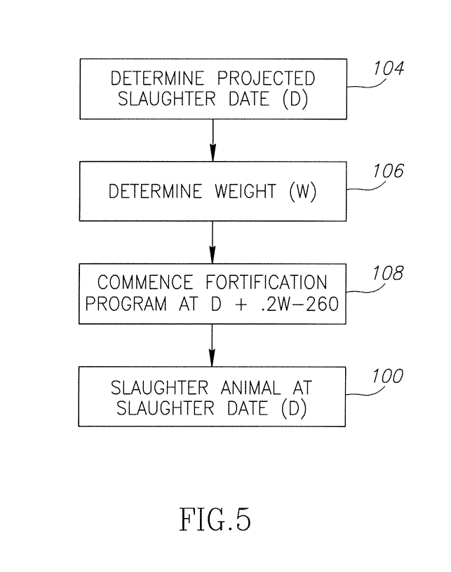

Referring to FIG. 5, In addition, or instead of, the above program, the daily ration fed to the offspring 16 may be fortified according to a novel fortification program discussed below. In one embodiment, cattle, such as the offspring 16, are fed a ration fortified with omega-3 fatty acids during a finishing period prior to slaughter. The fortification program may be accomplished by means of flax seed, flax seed oil, fishmeal or other omega-3 enrichener.

The fortification program is performed during a finishing period prior to slaughter in order to improve the results obtained relative to the amount of omega-3 fortification consumed. The fortification program is typically performed when the cattle are between twelve and twenty-four months old. Preferably, the fortification program is performed when the cattle are between 18 and twenty-four months old. The daily ration may be fortified for a fortification period beginning between 100 and 150 days prior to slaughter of the cattle, depending on the weight of the cattle.

In the method of FIG. 5, a projected slaughter date (D) is determined at block 144. The projected slaughter date (D) is typically when the offspring 16 is between twenty-one and twenty-nine months old. At block 146, the weight of the offspring 16 is determined. The weight (W) of the offspring 16 is typically determined just prior to commencement of the fortification program. At block 148, the fortification program is commenced at a date equal to D+0.2*W−260. In alternative embodiments, the fortification period begins at a dates from D+0.15*W−260 to D+0.25*W+260. At block 146, the offspring is slaughtered at about the projected slaughter date (D).

According to the method of FIG. 5, cattle, weighing about 600 pounds are fed an omega-3-fortified daily ration from about 140 days prior to slaughter. Cattle weighing about 800 pounds are fed an omega-3-fortified ration from about 100 days prior to slaughter.

Significant benefit may also be obtained for shorter periods including from one month to 60 days before slaughter. Furthermore, in some embodiments, significant benefits may still be obtained where the fortification period ends a short time such as from one week to one month before slaughter. In the preferred embodiment, the fortification ends substantially at slaughter, such as within the last few feedings before slaughter.

An omega-3 fortified ration is typically given to the offspring during two to three daily feedings. The omega-3-rich feed used may make up about five to fifteen percent of the caloric content of the daily ration. In a preferred embodiment, the omega-3-rich feed is equal to about ten percent of the caloric content of the daily ration.

Experiments conducted by the inventor have shown that cattle bred according to the novel breeding program and fed according to the novel omega-3 fortification program described above had surprisingly high omega-3 fat content. These results were obtained with the small investment of omega-3 enricheners described in the fortification program.

While preferred and alternate embodiments of the invention have been illustrated and described, as noted above, many changes can be made without departing from the spirit and scope of the invention. Accordingly, the scope of the invention is not limited by the disclosure of these preferred and alternate embodiments. Instead, the invention should be determined entirely by reference to the claims that follow.

What is claimed is:

1. A method of breeding beef cattle having an elevated omega-3 fatty acid content, comprising:
selecting a male bovine having a substantially Wagyu genetic composition;
selecting a female bovine having a genetic composition derived from selected other breeds;
producing an offspring by combining the genetic composition of the male bovine with the genetic composition of the female bovine; and
feeding the offspring a daily ration comprising an omega-3 enrichener during a fortification period.

2. The method of claim 1, wherein the omega-3 enrichener forms from five to fifteen percent of the energy content of the daily ration.

3. The method of claim 2, wherein the omega-3 enrichener forms ten percent of the energy content of the daily ration.

4. The method of claim 1, wherein the fortification period begins less than 150 days prior to slaughtering the offspring.

5. The method of claim 4, wherein the duration of the fortification period is a function of the weight of the offspring.

6. The method of claim 5, wherein the duration of the fortification period varies linearly according to weight of the offspring such that if the offspring weighs about 600 pounds the fortification period is greater than about 140 days and if the offspring weighs about 800 pounds the fortification period is greater than about 100 days.

7. The method of claim 4, wherein the duration of the fortification period is greater than about 260−0.2*W, where W is the weight of the offspring.

8. The method of claim 4, wherein the duration of the fortification period is greater than about 100 and less than about 150 days.

9. The method of claim 8, wherein the duration of the fortification period is less than 260-0.15*W and greater than 260-0.25*W where W is the weight of the offspring.

10. A method of raising beef cattle having an elevated omega-3 fatty acid content, comprising:
providing a bovine aged between twelve and twenty-four months;
feeding the bovine a daily ration comprising an omega-3 enrichener forming a substantial portion of the energy content of the daily ration for a fortification period of less than about 150 days and greater than 100 days; and
slaughtering the bovine proximate the ending of the fortification period.

11. The method of claim 10, wherein the fortification period ends at least one month before slaughter.

12. The method of claim 11, wherein the fortification period ends at least one week before slaughter.

13. The method of claim 12, wherein the fortification period ends substantially at slaughter.

14. The method of claim 10, wherein the omega-3 enrichener forms from five to fifteen percent of the energy content of the daily ration.

15. The method of claim 14, wherein the omega-3 enrichener forms ten percent of the energy content of the daily ration.

16. The method of claim 15, wherein the fortification period begins less than 150 days prior to slaughtering the bovine.

17. The method of claim 16, wherein the duration of the fortification period decreases with weight of the bovine.

18. The method of claim 17, wherein the duration of the fortification period varies linearly according to weight of the bovine such that if the bovine weighs about 600 pounds the fortification period is about 140 days and if the bovine weighs about 800 pounds the fortification period is about 100 days.

19. The method of claim 18, wherein the duration of the fortification period is equal to about 0.2*W−20, where W is the weight of the bovine.

20. The method of claim 10, wherein the bovine is an offspring of bull having a substantially Wagyu genetic composition and a cow having a genetic composition derived from selected other breeds.

21. The method of claim 20, wherein the bull has a Tajima Wagyu genetic composition.

22. The method of claim 20, wherein the bull has a genetic composition selected from the Tottori, Shimane and Okayama genetic compositions.

23. The method of claim 20, wherein he bull has a genetic composition selected from the Kochi and Kumamoto genetic compositions.

24. The method of claim 20, wherein the bull has a genetic composition selected from the Fukutsuru, Michifuku, Takazakura and Yasufuku genetic compositions.

25. The method of claim 20, wherein the bull is a male offspring resulting from a combination of a sire selected from the group consisting of the Fukutsuru, Michifuku, Takazakura and Yasufuku genetic compositions, and a dam selected from the group consisting of the Suzutani and Okutani genetic compositions.

26. The method of claim 25, wherein the bull has a genetic composition selected from the Sanjirou 3 and Yudai genetic compositions.

27. The method of claim 20, wherein the cow has a genetic composition of an English breed.

28. The method of claim 27, wherein the cow has a genetic composition selected from the Angus, Hereford, Short Horn, Devon and Ayrshire genetic compositions.

29. The method of claim 28, wherein the cow has a genetic composition of an English breed that produces milk having a high milk fat content.
